(19) United States
(12) Patent Application Publication
(10) Pub. No.: US 2003/0035833 A1
(43) Pub. Date: Feb. 20, 2003

(54) Rapidly dispersing pharmaceutical composition

(76) Inventor: Xiaorong He, Portage, MI (US)

Correspondence Address: Pharmacia Corporation, Patent Department, 800 N. Lindbergh Boulevard - #04E, St. Louis, MO, 63161, US

(21) Appl. No.: 10/008,223

(22) Filed: Dec. 5, 2001

Related U.S. Application Data
(60) Provisional application No. 60/251,694, filed on Dec. 6, 2000.

(51) Int. Cl. A61K 9/46

(52) U.S. Cl. 424/466

(57) ABSTRACT

A novel method is provided for enhancing dispersion of drug-containing particles in an aqueous medium. According to this method, a solid dosage form of the drug is provided having incorporated therein a dispersion-enhancing amount of an effervescent agent wherein (a) the dosage form is adapted for swallowing without prior disintegration in water or in the mouth, and (b) the amount of the effervescent agent is not sufficient to substantially enhance disintegration of the dosage form in the aqueous medium.

This application claims priority of U.S. provisional application Serial No. 60/251,694 filed Dec. 6, 2000.

### FIELD OF THE INVENTION

The present invention relates to orally deliverable solid pharmaceutical compositions, and in particular to such compositions that exhibit an enhanced rate of dispersion in an aqueous medium, for example gastrointestinal fluid.

### BACKGROUND OF THE INVENTION

Effervescent pharmaceutical compositions such as effervescent tablets are well known in the art. Generally, effervescent tablets consist of an active drug and a large fraction, generally greater than about 60% by weight of the total tablet, of an effervescent agent which typically comprises an acid source and a carbonate source. See, for example, Lieberman et al., ed. (1989), Pharmaceutical Dosage Forms: Tablets, Volume 1, 2nd ed., pp. 285-328. Marcel Dekker, New York. Although some effervescent tablets are designed to disintegrate in the mouth, most commonly effervescent tablets, for example Alka-Seltzer® effervescent tablets of Bayer Inc., are added to an aqueous medium such as water prior to oral administration, resulting in the formation of a solution or suspension and the evolution of carbon dioxide (or in some cases, oxygen) gas. This generation of gas promotes disintegration of the tablet in the aqueous medium, and the resulting solution or suspension is then imbibed after the tablet has more or less completely disintegrated. Such a method of administration can be advantageous, for example for patients who are unwilling or unable to swallow pills, or to provide a rapid onset of therapeutic effect since the process of tablet disintegration has already taken place prior to ingestion of the drug.

However, this method of administration is highly inconvenient in many situations since water is not always readily available throughout the day. Further, many drugs have a bitter taste that often cannot be masked even by the organoleptic enhancement or “mouth feel” characteristic of the sparkling solution or suspension provided by effervescent tablets when added to water. Additionally, preparation of such effervescent tablets requires special and costly processing conditions. For example, low relative humidity and moderate-to-cool temperatures are required in processing areas to prevent a granulated blend, or effervescent tablets prepared therefrom, from sticking to machinery and from picking up moisture from the air. Additionally, extra steps are often required, for example addition of special solvents, during processing to prevent the components of the effervescent agent, typically an acid and a base, from reacting. For these and other reasons, therefore, a solid dosage form that is swallowed prior to disintegration in water or in the mouth is generally preferred to an effervescent tablet.

The emergence of an orally administered drug (which is swallowed prior to disintegration in the mouth or in water) into systemic circulation depends on at least two fundamental processes: drug dissolution in gastrointestinal fluids (in vivo drug release) and subsequent absorption of the dissolved drug. Several factors influence dissolution of a drug substance from its carrier including surface area of the drug presented to the dissolution solvent medium, driving forces of the saturation concentration of dissolved materials in the solvent medium, and solubility of the drug substance in the specific solvent medium. Notwithstanding these factors, a strong correlation has been established between the in vitro dissolution time determined for a dosage form and the rate of in vivo drug release. This correlation is so firmly established in the art that dissolution time has become generally descriptive of drug release potential for the active component of the particular unit dosage composition.

When the process of in vivo drug release is slower than the process of absorption, absorption is said to be dissolution rate-limited. Since dissolution precedes absorption in the overall process, any change in the drug release or dissolution process will subsequently influence drug absorption. Lieberman et al., op. cit., Vol. 1, pp. 34-36. It is clear, therefore, that the dissolution time determined for a composition is one of the important fundamental characteristics for consideration when evaluating rapid-onset compositions, particularly where drug absorption is dissolution rate-limited.

Many pharmaceutically useful drugs have low solubility in water and other aqueous media. Even after disintegration of an oral dosage form containing such a drug, the drug tends not to disperse, but to aggregate together. This poor dispersion, for example when occurring in gastrointestinal fluids, leads to slow drug dissolution and, subsequently, to decreased absorption and therefor poor bioavailability.

Measures to increase solubility of hydrophobic, crystalline drugs (e.g., by adding conventional wetting agents, by dispersing the drug in solid matrices, by preparing amorphous drug particles, by decreasing drug particle size, etc.) have been attempted in hopes of improving drug dissolution characteristics; however, these attempts have achieved only limited success. Drug particles, even following such measures, still tend to aggregate together upon contact with aqueous fluids such as those of the gastrointestinal tract, the resulting poor dispersion tending to offset any advantage of improved dissolution.

Therefore, if a solid dosage form comprising a drug of low water solubility, which dosage form exhibits increased drug dispersion in aqueous media, could be developed, a significant advantage would be realized in the utility of drugs, particularly those of low solubility, and more particularly those used to treat disorders where rapid onset of therapeutic effect is desired.

### SUMMARY OF THE INVENTION

Accordingly, the present invention provides a method for enhancing dispersion of drug-containing particles in an aqueous medium, the method comprising providing a solid dosage form of the drug having incorporated therein a dispersion-enhancing amount of an effervescent agent wherein (a) the dosage form is adapted for swallowing without prior disintegration in water or in the mouth, and (b) the amount of the effervescent agent is not sufficient to substantially enhance disintegration of the dosage form in the aqueous medium.

Typically but without limitation, a suitable dispersion-enhancing amount of the effervescent agent is about 1% to about 20% by weight of the dosage form.

The invention also provides in one embodiment a solid pharmaceutical composition comprising a therapeutically and/or prophylactically effective amount of a drug and a dispersion-enhancing amount of an effervescent agent wherein (a) the dosage form is adapted for swallowing without prior disintegration in water or in the mouth, and (b) the amount of the effervescent agent is not sufficient to substantially enhance disintegration of the dosage form in an aqueous medium.

A dosage form which is “adapted for swallowing without prior disintegration in water or in the mouth” is preferably, among other properties, of a size that is not so large that it is impossible, uncomfortable or difficult to be swallowed whole. In a preferred embodiment, therefore, the dosage form has a total weight no greater than about 800 mg, for example about 50 mg to about 800 mg. More preferably the dosage form has a total weight of about 100 mg to about 750 mg, most preferably about 200 mg to about 700 mg.

Accordingly, therefore, the invention provides in another embodiment a solid pharmaceutical dosage form comprising a therapeutically and/or prophylactically effective amount of a drug and a dispersion-enhancing amount of an effervescent agent, wherein the dosage form does not exceed about 800 mg in total weight. In this embodiment the amount of the effervescent agent may or may not be sufficient to substantially enhance disintegration of the dosage form in an aqueous medium.

Also provided are processes for preparing compositions and dosage forms of the invention. One illustrative process comprises (a) providing a drug in finely divided form; (b) admixing the finely divided drug with an effervescent agent and optionally with one or more pharmaceutically acceptable excipients to form a mixture; and (c) applying mechanical means to the mixture to form a drug powder wherein the drug and the effervescent agent are in intimate association. Optionally, the process can further comprise (d) blending the drug powder with one or more excipients to form a blend; and (e) compressing or encapsulating the blend to form tablets or capsules respectively.

### DETAILED DESCRIPTION OF THE INVENTION

Disintegration and Dispersion

Disintegration of a solid dosage form such as a tablet, caplet or capsule, with respect to both extent and time, can be measured using a standard United States Pharmacopeia (USP) disintegration assay. In this assay, an apparatus is employed that consists of a basket-rack assembly containing a number of open-ended glass tubes held vertically upon a stainless steel wire mesh screen. During testing, a dosage form is placed in each tube and a mechanical device raises and lowers the basket in an immersion fluid, usually water at 37° C., at a frequency of about 29 to about 32 immersion cycles per second. Complete disintegration of a solid dosage form is observed when none of the residue of the dosage form, except fragments of insoluble coating or capsule shell, remain on the screen of the test apparatus.

As used herein, the phrase “an amount not sufficient to substantially enhance disintegration of the dosage form” in reference to the amount of effervescent agent present, indicates an amount less than that which will substantially speed up, enhance, expedite, affect, facilitate or promote disintegration as measured in a standard USP disintegration assay.

The term “dispersion” as used herein refers to the process by which a disintegration residue (including but not limited to granules, aggregates or particles) which is formed from disintegration of a solid composition in an aqueous medium as described above, separates or de-aggregates to form fine particles. To “enhance dispersion” as described herein means to cause, increase, facilitate or promote dispersion. Rate and extent of dispersion can be measured by aided (e.g., by microscope, etc.) or unaided visual observation, by filtration, or by any other suitable means.

The term “dissolution” as used herein refers to the process by which a solid enters into solution.

Drug

Any suitable drug may be utilized in methods, processes and compositions of the invention. Preferably, the drug is one having low water solubility, for example a solubility in water, measured at 37° C., not greater than about 10 mg of drug per ml of water, and preferably not greater than about 1 mg of drug per ml of water. Solubility in water for many drugs can be readily determined from standard pharmaceutical reference books, for example The Merck Index, 11th ed., 1989 (published by Merck & Co., Inc., Rahway, N.J.); the United States Pharmacopoeia, 24th ed. (USP 24), 2000; The Extra Pharmacopoeia, 29th ed., 1989 (published by Pharmaceutical Press, London); and the Physicians Desk Reference (PDR), 2000 ed. (published by Medical Economics Co., Montvale, N.J.), each of which is individually incorporated herein by reference.

For example, individual drugs of low solubility as defined herein include those drugs categorized as “slightly soluble”, “very slightly soluble”, “practically insoluble” and “insoluble” in USP 24, pp. 2254-2298; and those drugs categorized as requiring 100 ml or more of water to dissolve 1 g of the drug, as listed in USP 24, pp. 2299-2304.

Illustratively, suitable drugs of low water solubility include, without limitation, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I annd II inhibitors, uricosurics, vasodilators, vasoprotectants, xanthine oxidase inhibitors, and combinations thereof.

Non-limiting illustrative examples of suitable drugs of low water solubility include, for example, acetylsalicylic acid, allopurinol, acetohexamide, atropine, benzthiazide, diclofenac, alclofenac, fenclofenac, etodolac, indomethacin, sulindac, tolmetic, fentiazac, tilomisole, carpofen, fenbufen, flurbiprofen, ketoprofen, oxaprozin, suprofen, tiaprofenic acid, ibuprofen, naproxen, fenprofen, indoprofen, pirprofen, niflumic, celecoxib, chlorpromazine, chlordiazepoxide, clonidine, codeine, codeine sulfate, codeine phosphate, deracoxib, diacerein, diltiazem, enolic acids, estradiol, etoposide, griseofulvin, haloperidol, indomethacine, lorazepam, methoxsalen, methylprednisone, megestrol, medroxyprogesterone acetate, morphine, morphine sulfate, nicergoline, nifedipine, oxazepam, oxyphenbutazone, parecoxib, phenobarbital, phenindione, piroxicam, prednisone, prednisolone, progesterone, procaine, pyrimethamine, rofecoxib, sulfadiazine, sulfisoxazole, sulfamerazine, temazepam, valdecoxib, etc.

The amount of drug incorporated in a dosage form of the invention can be selected according to known principles of pharmacy. A therapeutically effective amount of drug is specifically contemplated. The term “therapeutically and/or prophylactically effective amount” as used herein refers to an amount of drug which is sufficient to elicit the required or desired therapeutic and/or prophylactic response.

Effervescent Agent

An “effervescent agent” herein is an agent comprising one or more compounds which, acting together or individually, evolve a gas on contact with water. The gas evolved is generally oxygen or, most commonly, carbon dioxide. Preferred effervescent agents comprise an acid component and a base component that react in the presence of water to generate carbon dioxide gas. The acid component can comprise one or more acids and the base component can comprise one or more bases. Preferably, the base component comprises an alkali metal or alkaline earth metal carbonate or bicarbonate and the acid component comprises an aliphatic carboxylic acid.

Non-limiting examples of suitable bases for use in a base component include carbonate salts (e.g., calcium carbonate), bicarbonate salts (e.g., sodium bicarbonate), sesquicarbonate salts, and mixtures thereof. Calcium carbonate is a preferred base.

Non-limiting examples of suitable acids for use in an acid component include citric acid, tartaric acid, malic acid, fumaric acid, adipic acid, succinic acid, acid anhydrides of such acids, acid salts of such acids, and mixtures thereof. Citric acid is a preferred acid.

In a preferred embodiment of the invention, where the effervescent agent comprises an acid component and a base component, the weight ratio of the acid component to the base component is about 1:100 to about 100:1, more preferably about 1:50 to about 50:1, and still more preferably about 1:10 to about 10:1. In a further preferred embodiment of the invention, where the effervescent agent comprises an acid component and a base component, the ratio of the acid component to the base component is approximately stoichiometric.

Because it is useful for a dosage form of the invention to be small enough to be comfortably swallowed whole, it is preferred that the drug loading in the dosage form be as high as possible, especially where the therapeutically effective dose is fairly high. In a particularly preferred embodiment, therefore, the amount of effervescent agent present, as a fraction of the total weight of the dosage form, is small enough to allow a therapeutically effective dose of the particular drug to be incorporated into a dosage form no greater than about 800 mg in total weight. Typically, according to this embodiment, the amount of effervescent agent is not greater than about 20% by weight of the dosage form.

An effervescent agent as defined above is preferably present in a composition of the invention in an amount of about 1% to about 20%, more preferably about 2% to about 15% and still more preferably about 3% to about 10%, by weight of the composition. As indicated herein, the amount of the effervescent agent is not sufficient to provide substantial enhancement of disintegration of the composition, but in accordance with the invention surprisingly is sufficient to provide substantial enhancement of dispersion of primary particles of the composition in an aqueous medium. Preferably, such enhanced dispersion is accompanied by substantial enhancement of rate of dissolution of the drug in the aqueous medium.

Excipients

Solid pharmaceutical compositions of the invention can further comprise one or more excipients other than the effervescent agent. The term “excipient” herein means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients include, by way of illustration and not limitation, diluents, disintegrants, binding agents, adhesives, wetting agents, lubricants, glidants, crystallization inhibitors, surface modifying agents, substances added to mask or counteract a disagreeable taste or odor, flavors, dyes, fragrances, and substances added to improve appearance of the composition.

Excipients employed in compositions of the invention can be solids, semi-solids, liquids or combinations thereof. Compositions of the invention containing excipients can be prepared by any known technique of pharmacy that comprises admixing an excipient with a drug or therapeutic agent.

Non-limiting examples follow of excipients that can be used to prepare pharmaceutical compositions of the invention.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone (PVP); and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. Both diluents are chemically compatible with celecoxib. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of celecoxib, stability, pre-compression flowability, and/or drying properties at a relatively low diluent cost. It provides a high density substrate that aids densification during granulation (where wet granulation is employed) and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone (polyvinylpyrrolidone, PVP), for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Such wetting agents are preferably selected to maintain the celecoxib in close association with water, a condition that is believed to improve bioavailability of the composition.

Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

Process for Making Compositions of the Invention

Solid pharmaceutical compositions of the invention can be prepared by any suitable process, not limited to processes described herein. An illustrative process for preparing a composition of the invention comprises (a) providing a drug in finely divided form; (b) admixing the finely divided drug with an effervescent agent and optionally with one or more pharmaceutically acceptable excipients to form a mixture; and (c) applying mechanical means to the mixture to form a drug powder wherein the drug and the effervescent agent are in intimate association. Optionally, this process can further comprise (d) a step of blending the drug powder with one or more excipients to form a blend; and (e) a step of compressing or encapsulating the blend to form tablets or capsules, respectively.

A “finely divided drug” herein is a drug substance or a composite thereof with one or more excipients such as a polymer, the drug substance or composite being in the form of particles in the micro- or nanometer size range (e.g., having a weight average particle size of about 0.01 μm to about 100 μm, preferably about 0.1 μm to about 10 μm).

Mechanical Means to Form Drug Powder

Any suitable mechanical means can be applied to prepare drug powders in processes of the invention. Non-limiting examples of suitable mechanical means include milling (e.g., ball milling, McCrone milling, pin milling, etc.), grinding, spray drying, granulating, blending, etc. It is preferred that where granulation is used as the mechanical means, the effervescent agent is incorporated intragranularly as opposed to extragranularly. Preparation of the drug powder is conducted substantially in the absence of water to prevent premature reaction of the effervescent agent. Where processes involving a liquid are used, such as wet granulation or spray drying, a suitable non-aqueous liquid is employed. However, it is preferred that the mechanical means for preparing the drug powder be conducted substantially in the absence of liquid.

A drug powder or blend prepared by any of the above illustrative means can be compressed (to prepare tablets) or encapsulated (to prepare capsules). Conventional compression and encapsulation techniques known to those of ordinary skill in the art can be employed. Where coated tablets are desired, conventional coating techniques are suitable.

Excipients for tablet compositions of the invention preferably are selected to provide a disintegration time of less than about 30 minutes, preferably about 25 minutes or less, more preferably about 20 minutes or less, and still more preferably about 15 minutes or less, in a standard disintegration assay.

Any tablet hardness convenient with respect to handling, manufacture, storage and ingestion may be employed. For 100 mg tablets, hardness is preferably at least 4 kP, more preferably at least about 5 kP, and still more preferably at least about 6 kP. For 200 mg tablets, hardness is preferably at least 7 kP, more preferably at least about 9 kP, and still more preferably at least about 11 kP. The mixture, however, is not to be compressed to such a degree that there is subsequent difficulty in achieving hydration when exposed to gastric fluid.

Tablet friability preferably is less than about 1.0%, more preferably less than 0.8%, and still more preferably less than about 0.5% in a standard test.

### EXAMPLES

The following examples illustrate aspects of the present invention but should not be construed as limitations. While celecoxib is used as the drug in these examples, it will be understood that the invention can be practiced with any drug, particularly a drug of low water solubility.

### Example 1

Drug powders D1-D7 having the ingredients set out in Table 1 below were prepared according to the following process.

1. Crystalline celecoxib in the amount of 30 mg was dissolved in 2000 ml 95% ethanol containing 15 mg/ml PVP, at a temperature of 70-75° C. with stirring, to form solution S1.

2. Solution S1 was spray dried at room temperature using a Yamato GB-21 spray dryer to form a celecoxib composite under the following conditions: (a) liquid flow rate of 10 ml/min; (b) inlet air temperature of 115° C.; (c) outlet air temperature of 75° C., and (d) a drying airflow of about 30% to about 50% of the capacity of the spray dryer.

3. A known weight of the resulting celecoxib composite was admixed together with either a non-effervescent disintegrant (sodium lauryl sulfate) or with an effervescent agent (sodium bicarbonate and citric acid anhydrous) in amounts shown in Table 1 to form mixtures.

4. The resulting mixtures were either (a) milled for 10 minutes in a McCrone mill (D2-D7) or (b) ground with a mortar and pestle (D1) to form drug powders.

TABLE 1
Components (weight %) of drug powders D1-D7

| Component | D1 | D2 | D3 | D4 | D5 | D6 | D7 |
|---|---|---|---|---|---|---|---|
| Celecoxib composite | 100 | 99 | 62 | 87 | 91 | 94 | 96.6 |
| Sodium lauryl sulfate | — | 1 | 1 | 1 | 1 | 1 | 1 |
| Sodium bicarbonate | — | — | 16 | 7.4 | 5 | 3 | 1.4 |
| Citric acid anhydrous | — | — | 21 | 4.6 | 3 | 2 | 1 |
| Total | 100 | 100 | 100 | 100 | 100 | 100 | 100 |

### Example 2

Drug powders D1-D7 were evaluated in an in vitro dispersion assay. In this assay, 1 mg of each drug powder was individually placed into a beaker containing 100 ml of deionized water. Liquid aliquots were then immediately withdrawn and viewed under the microscope to evaluate for particle dispersion and clumping. Observations are shown in Table 2, below.

TABLE 2
In vitro dispersion of drug powders D1-D7

| Drug powder | Observation |
|---|---|
| D1 | Large clumps (200-2000 μm) which do not disperse with |
| | shaking or stirring |
| D2 | Small clumps (10-200 μm) which do not disperse with |
| | shaking or stirring |
| D3 | Instantaneous fine dispersion |
| D4 | Instantaneous fine dispersion |
| D5 | Fine dispersion within a few seconds, with slight shaking |
| D6 | Fine dispersion within a few seconds, with slight shaking |
| D7 | Fine dispersion within 75 seconds, with no shaking |

### Example 3

Three powder blends, B1, B2 and B3 were prepared by grinding or milling a drug powder prepared as in Example 1 or a drug powder comprising the celecoxib composite of Example 1 and sodium lauryl sulfate, together with additional excipients. Compositions of the powder blends are shown in Table 3, below.

TABLE 3
Composition (mg) of powder blends B1-B3

| Component | B1¹ | B2² | B3² |
|---|---|---|---|
| Drug powder D2 | — | 300 | — |
| Drug powder D4 | 30 | — | — |
| Celecoxib composite | — | — | 300 |
| Sodium lauryl sulfate | — | — | 3 |
| Citric acid anhydrous | — | 16 | — |
| Sodium bicarbonate | — | 25 | — |
| Lactose | 10 | 107 | 100 |
| Microcrystalline cellulose | 5 | 52 | 50 |
| Sodium starch glycolate | 4 | 40 | 40 |
| Total | 49 | 540 | 493 |

¹milled;
²ground.

### Example 4

Powder blends B1-B3 were evaluated in the in vitro dispersion assay described in Example 2. Observations are shown in Table 4, below. Powder blend B1 that was prepared from drug powder D4 having an effervescent agent incorporated therein dispersed faster than powder blend B2 that was prepared from drug powder D2 ground together with effervescent agent. Blend B2 containing an effervescent agent dispersed much better than did blend B3 containing no effervescent agent.

TABLE 4
In vitro dispersion assay of powder blends B1-B3

| Powder blend | Observation |
|---|---|
| B1 | Instantaneous dispersion |
| B2 | Dispersion within 40 seconds |
| B3 | Incomplete dispersion after 10 minutes |

### Example 5

Four tablet prototypes T1-T4 were prepared in order to compare disintegration and dispersion of solid dosage forms containing an effervescent agent with those containing no effervescent agent. Drug powder D4 of Example 1 was (a) mixed with a non-effervescent disintegrant only (T3), (b) mixed with sodium starch glycolate and an effervescent agent (T2), or (c) mixed with an effervescent agent only (T1), to form powder blends. Further, a control powder blend comprising celecoxib composite prepared as in Example 1 and other excipients (but no effervescent agent) was also prepared (T4). All powder blends were ground in a mortar and pestle for 3 minutes. An amount of 500 or 600 mg of each powder blend was compressed using a Carver press at around 900 kg. Tablet tooling was externally lubricated with magnesium stearate prior to compression. Components of powder blends used to make tablet prototypes T1-T4 are shown in Table 5, below.

TABLE 5
Components (mg) of tablet prototypes T1-T4

| Component | T1 | T2 | T3 | T4 |
|---|---|---|---|---|
| Celecoxib composite | — | — | — | 296.8 |
| Sodium lauryl sulfate | — | — | — | 1.3 |
| Drug powder D4 | 345 | 345 | 345 | — |
| Lactose | — | 70 | 50 | 107.2 |
| Microcrystalline cellulose | — | 77 | 57 | 52.3 |
| Sodium starch glycolate | — | 30 | 48 | 40 |
| Citric acid anhydrous | 100 | 30 | — | — |
| Sodium bicarbonate | 155 | 48 | — | — |
| Magnesium stearate | — | — | — | 22.5 |
| Total | 600 | 600 | 500 | 500 |
| % effervescent agent | 49 | 20 | 8 | 0 |

### Example 6

Tablet prototypes T1-T4 were evaluated individually in a USP disintegration assay. The apparatus consisted of a basket-rack assembly, a 1000 ml beaker for the immersion fluid, a thermostatic arrangement for heating the fluid and a device for raising and lowering the basket in the immersion fluid at a constant frequency of 29 to 32 cycles. The fluid temperature was around 37° C.; either a 20-mesh or 40-mesh screen was used for the basket. Disintegration time was counted as the time for all tablet residues passing through the screen.

Dispersion of tablet prototypes T1-T4 was observed as described in Example 2, above.

Results are shown in Table 6, below.

TABLE 6
Disintegration and dispersion of tablet prototypes T1-T4

| Tablet prototype | Disintegration time (min) | Dispersion observation |
|---|---|---|
| T1 | >7¹ | Fine particle dispersion |
| T2 | 3² | Fine particle dispersion |
| T3 | 3.7² | Fine particle dispersion |
| T4 | 2.5^{2,3} | Small chunks |

¹40 mesh screen;
²20 mesh screen;
³small amount of residue remaining on screen.

Overall, these observations indicate that neither Tablet T2 nor Tablet T3 of the present invention have sufficient effervescent agent to substantially enhance tablet disintegration as compared to Tablet T4 comprising no effervescent agent, yet both T2 (containing 20% effervescent agent) and T3 (containing 8% effervescent agent) exhibited enhanced drug dispersion in vitro.

What is claimed is:

1. A method for enhancing dispersion of drug-containing particles in an aqueous medium, the method comprising providing a solid dosage form of the drug having incorporated therein a dispersion-enhancing amount of an effervescent agent, wherein (a) the dosage form is adapted for swallowing without prior disintegration in water or in the mouth, and (b) the amount of the effervescent agent is not sufficient to substantially enhance disintegration of the dosage form in the aqueous medium.

2. The method of claim 1 wherein the drug is of low water solubility.

3. The method of claim 1 wherein the rate of dissolution of the drug in the aqueous medium is enhanced.

4. The method of claim 1 wherein the effervescent agent generates oxygen or carbon dioxide gas upon contact with water.

5. The method of claim 1 wherein the dosage form is selected from the group consisting of a tablet, caplet, capsule, drug powder or powder blend.

6. The method of claim 1 wherein the effervescent agent comprises an acid component and a base component.

7. The method of claim 6 wherein the acid component comprises at least one acid selected from the group consisting of citric acid, tartaric acid, malic acid, fumaric acid, adipic acid, succinic acid, acid anhydrides and acid salts thereof, and mixtures thereof.

8. The method of claim 7 wherein the at least one acid is citric acid.

9. The method of claim 6 wherein the base component comprises at least one base selected from the group consisting of carbonate salts, bicarbonate salts, sesquicarbonate salts, and mixtures thereof.

10. The method of claim 9 wherein the at least one base is calcium carbonate.

11. The method of claim 6 wherein the weight ratio of the acid component to the base component in the effervescent agent is about 1:100 to about 100:1.

12. The method of claim 6 wherein the weight ratio of the acid component to the base component in the effervescent agent is about 1:50 to about 50:1.

13. The method of claim 6 wherein the weight ratio of the acid component to the base component in the effervescent agent is about 1:10 to about 10:1.

14. The method of claim 6 wherein the ratio of the acid component to the base component in the effervescent agent is approximately stoichiometric.

15. The method of claim 1 wherein the effervescent agent is present in the dosage form in an amount of about 1% to about 20% by weight.

16. The method of claim 1 wherein the effervescent agent is present in the dosage form in an amount of about 2% to about 15% by weight.

17. The method of claim 1 wherein the effervescent agent is present in the dosage form in an amount of about 3% to about 10% by weight.

18. A solid pharmaceutical composition comprising a therapeutically and/or prophylactically effective amount of a drug and a dispersion-enhancing amount of an effervescent agent, wherein (a) the dosage form is adapted for swallowing without prior disintegration in water or in the mouth, and (b) the amount of the effervescent agent is not sufficient to substantially enhance disintegration of the dosage form in an aqueous medium.

19. The composition of claim 18 wherein the drug is of low water solubility.

20. The composition of claim 18 wherein the rate of dissolution of the drug in an aqueous medium is enhanced.

21. The composition of claim 18 wherein the effervescent agent generates oxygen or carbon dioxide gas upon contact with water.

22. The composition of claim 18 that is a dosage form selected from the group consisting of a tablet, a caplet, a capsule, a drug powder and a powder blend.

23. The composition of claim 18 wherein the effervescent agent comprises an acid component and a base component.

24. The composition of claim 23 wherein the acid component comprises at least one acid selected from the group consisting of citric acid, tartaric acid, malic acid, fumaric acid, adipic acid, succinic acid, acid anhydrides and acid salts thereof, and mixtures thereof.

25. The composition of claim 24 wherein the at least one acid is citric acid.

26. The composition of claim 23 wherein the base component comprises at least one base selected from the group consisting of carbonate salts, bicarbonate salts, sesquicarbonate salts, and mixtures thereof.

27. The composition of claim 26 wherein the at least one base is calcium carbonate.

28. The composition of claim 23 wherein the weight ratio of the acid component to the base component in the effervescent agent is about 1:100 to about 100:1.

29. The composition of claim 23 wherein the weight ratio of the acid component to the base component in the effervescent agent is about 1:50 to about 50:1.

30. The composition of claim 23 wherein the weight ratio of the acid component to the base component in the effervescent agent is about 1:10 to about 10:1.

31. The composition of claim 23 wherein the ratio of the acid component to the base component in the effervescent agent is approximately stoichiometric.

32. The composition of claim 18 wherein the effervescent agent is present in the composition in an amount of about 1% to about 20% by weight.

33. The composition of claim 18 wherein the effervescent agent is present in the composition in an amount of about 2% to about 15% by weight.

34. The composition of claim 18 wherein the effervescent agent is present in the composition in an amount of about 3% to about 10% by weight.

35. A solid pharmaceutical dosage form comprising a therapeutically and/or prophylactically effective amount of a drug and a dispersion-enhancing amount of an effervescent agent, wherein the dosage form does not exceed about 800 mg in total weight.

36. The dosage form of claim 35 wherein said dosage form has a total weight of about 100 to about 750 mg.

37. The dosage form of claim 35 wherein said dosage form has a total weight of about 200 to about 700 mg.

38. The composition of claim 35 wherein the drug is of low water solubility.

39. The composition of claim 35 wherein the rate of dissolution of the drug in an aqueous medium is enhanced.

40. The composition of claim 35 wherein the effervescent agent generates oxygen or carbon dioxide gas upon contact with water.

41. The composition of claim 35 that is a dosage form selected from the group consisting of a tablet, a caplet, a capsule, a drug powder and a powder blend.

42. The composition of claim 35 wherein the effervescent agent comprises an acid component and a base component.

43. The composition of claim 42 wherein the acid component comprises at least one acid selected from the group consisting of citric acid, tartaric acid, malic acid, fumaric acid, adipic acid, succinic acid, acid anhydrides and acid salts thereof, and mixtures thereof.

44. The composition of claim 43 wherein the at least one acid is citric acid.

45. The composition of claim 42 wherein the base component comprises at least one base selected from the group consisting of carbonate salts, bicarbonate salts, sesquicarbonate salts, and mixtures thereof.

46. The composition of claim 45 wherein the at least one base is calcium carbonate.

47. The composition of claim 42 wherein the weight ratio of the acid component to the base component in the effervescent agent is about 1:100 to about 100:1.

48. The composition of claim 42 wherein the weight ratio of the acid component to the base component in the effervescent agent is about 1:50 to about 50:1.

49. The composition of claim 42 wherein the weight ratio of the acid component to the base component in the effervescent agent is about 1:10 to about 10:1.

50. The composition of claim 42 wherein the ratio of the acid component to the base component in the effervescent agent is approximately stoichiometric.

51. The composition of claim 35 wherein the effervescent agent is present in the composition in an amount of about 1% to about 20% by weight.

52. The composition of claim 35 wherein the effervescent agent is present in the composition in an amount of about 2% to about 15% by weight.

53. The composition of claim 35 wherein the effervescent agent is present in the composition in an amount of about 3% to about 10% by weight.

54. A process for preparing a composition of claim 18, the process comprising
(a) providing the drug in finely divided form;
(b) admixing the finely divided drug with an effervescent agent and optionally with one or more pharmaceutically acceptable excipients to form a mixture; and
(c) applying mechanical means to the mixture to form a drug powder wherein the drug and the effervescent agent are in intimate association.

55. The process of claim 54 further comprising
(d) blending the drug powder with one or more excipients to form a blend; and
(e) compressing the blend to form tablets.

56. The process of claim 54 further comprising
(d) blending the drug powder with one or more excipients to form a blend; and
(e) encapsulating the blend to form capsules.

57. The process of claim 54 wherein the mechanical means is selected from the group consisting of milling, grinding, blending, spray drying and granulating.

58. A process for preparing a composition of claim 35, the process comprising
(a) providing the drug in finely divided form;
(b) admixing the finely divided drug with an effervescent agent and optionally with one or more pharmaceutically acceptable excipients to form a mixture; and
(c) applying mechanical means to the mixture to form a drug powder wherein the drug and the effervescent agent are in intimate association.

59. The process of claim 58 further comprising
(d) blending the drug powder with one or more excipients to form a blend; and
(e) compressing the blend to form tablets.

60. The process of claim 58 further comprising
(d) blending the drug powder with one or more excipients to form a blend; and
(e) encapsulating the blend to form capsules.

61. The process of claim 58 wherein the mechanical means is selected from the group consisting of milling, grinding, blending, spray drying and granulating.
